(19) United States
(12) Patent Application Publication
(10) Pub. No.: US 2019/0116829 A1
(43) Pub. Date: Apr. 25, 2019

(54) CHOCOLATE 3D PRINTING MATERIAL AND METHOD FOR PRODUCING THE SAME

(71) Applicant: INFINITUS (CHINA) COMPANY LTD., Jiang Men (CN)

(72) Inventors: Weihong CHEN, Jiang Men (CN); Junyong XIAO, Jiang Men (CN); Renhuai CONG, Jiang Men (CN); Fangli MA, Jiang Men (CN); Chung Wah MA, Jiang Men (CN)

(73) Assignee: INFINITUS (CHINA) COMPANY LTD., Jiang Men (CN)

(21) Appl. No.: 16/166,157

(22) Filed: Oct. 22, 2018

(30) Foreign Application Priority Data
Oct. 23, 2017 (CN) 201710993127.1

(51) Int. Cl. A23G 1/48; A23G 1/40; B33Y 70/00; B33Y 80/00; A61K 9/00; A61K 36/07; A61K 36/076; A61K 36/815; A61K 36/605; A61K 36/482; A61K 36/185; A61K 36/074; A61K 36/284

(52) U.S. Cl. CPC A23G 1/48; A23G 1/40; B33Y 70/00; B33Y 80/00; A61K 9/0056; A61K 36/07; A23V 2002/00; A61K 36/815; A61K 36/605; A61K 36/482; A61K 36/185; A61K 36/074; A61K 36/284; A61K 36/076

(57) ABSTRACT

The present disclosure relates to the field of food technology, specifically to a chocolate 3D printing material and a method for producing the same. In the chocolate 3D printing material provided by the present disclosure, the conventional chocolate sugar is replaced with maltitol and xylitol, which increases the fluidity of the chocolate paste while ensures the taste, and guarantees smooth discharge during 3D printing. The application of the traditional Chinese medicine extract may induce the formation of a stable crystal form V, promoting the mutual miscibility of chocolate multiphase system, preventing chocolate bloom, and extending the shell life of chocolate.

### CROSS REFERENCE TO RELATED APPLICATIONS

This application claims the priority of Chinese Patent Application No. 201710993127.1, filed on Oct. 23, 2017, and the disclosures of which are hereby incorporated by reference.

### FIELD

The present disclosure relates to the field of food technology, specifically to a chocolate 3D printing material and a method for producing the same.

### BACKGROUND

Chocolate is a kind of sweet food mainly made from cocoa. It is not only delicate and sweet but also has a strong aroma. Chocolate can be eaten directly or used to make cakes, ice creams, etc. In the early 16th century, the Spanish explorer Hernando Cortes discovered in Mexico that the local Aztec king had a drink made of cocoa beans with water and spices. Cortez brought it back to Spain in 1528 after he tasted it, and planted cocoa trees on a small island in West Africa. The Spanish made the cocoa beans into powder, adding water and sugar, and the beverage made after heating was called “chocolate”, which was well received by the public. In 1847, cocoa butter was added to the chocolate drink to make a chewable chocolate bar that is now well known.

Current chocolate is produced from cocoa beans, which are cleaned, screened, roasted, shelled, alkalized (or non-alkalized), and the paste is finely ground into a cocoa liquor, also known as cocoa material or bitter chocolate. The cocoa liquor has the characteristics of a fluid in a warm state, and solidifies into a block after cooling, so it is called a liquid block. Cocoa liquor is an important raw material for the production of chocolate. The cocoa liquor can be obtained by pressing to obtain cocoa butter and cocoa cake, and the cocoa cake is an essential raw material for processing into various cocoa powders.

Chocolate is popular among consumers all over the world for its delicate taste, rich and unique flavor. But at the same time, chocolate is a high-calorie food with high fat and sugar content and low protein content. Long-term drinking of chocolate is not conducive to health. Natural plant extracted polysaccharides have functions such as anti-tumor, anti-oxidation, promoting protein and nucleic acid synthesis, resisting radiation damage and increasing white blood cell content, anti-ulcer and anti-inflammatory, lowering blood sugar, lowering blood lipids, anti-thrombosis, liver protection, anticoagulant effect, and enhancing bone marrow hematopoietic function.

3D printing technology is based on the blueprint of computer three-dimensional design model, and the printing materials are stacked layer by layer through a software layer discretization and a computer numerical control system. Finally the printing materials are superposed to form a physical product. 3D printing is combined with individualization, according to the individual needs, the healthy chocolate with various natural active ingredients can be printed. Utilizing the developed 3D printer used for food may print the chocolate with various tastes, shapes and special health-care functions.

Currently, the chocolate with health-care ingredients made by 3D printing has not been sold on the market. The current products of chocolate with health-care ingredients mostly are stuffing structures, since the fluidity of the chocolate is not uniform after melting, it cannot be directly used for 3D printing. The viscosity of some chocolate paste is not in a proper range, which is too low, the low viscosity chocolate paste cannot form by printing. If the viscosity is too high, which requires larger mechanical driving force, and the high viscosity chocolate paste may even block the printing needle to stop the printing.

Some of pure cocoa chocolate used for 3D printing can be used for 3D printing after melting, since the chocolate printing has neither the tempering process for producing traditional chocolate nor the forming step of a seed crystal introduction to induce a stable crystal forms during manual preparation. Various crystal forms are mixed in the formed chocolate after printing, and there are several problems, like loose texture, poor taste, poor surface gloss, and the like.

### SUMMARY

In view of the above, the technical problem to be solved in the present disclosure is to provide a chocolate printing material for 3D printing. The chocolate paste includes natural active ingredients extracted from plants, has stable properties, may be well formed during the printing process and has a good taste and appearance after solidification.

The present disclosure provides a chocolate 3D printing material, which is made from the following raw materials in parts by mass:

| | |
|---|---|
| Cocoa liquor | 1495~1580 parts; |
| Cocoa butter | 712~800 parts; |
| Maltitol | 870~1260 parts; |
| Xylitol | 241~341 parts; |
| Lecithin | 16~28 parts; |
| Stevioside | 1~5 parts; |
| Traditional Chinese medicine extract | 0~600 parts. |

In some embodiments, the chocolate 3D printing materials comprises the following raw materials in parts by mass:

| | |
|---|---|
| Cocoa liquor | 1495~1580 parts; |
| Cocoa butter | 712~800 parts; |
| Maltitol | 870~960 parts; |
| Xylitol | 241~290 parts; |
| Lecithin | 16~28 parts; |
| Stevioside | 3~5 parts; |
| Traditional Chinese medicine extract | 400~600 parts. |

In some embodiments, the chocolate 3D printing material comprises the following raw materials in parts by mass:

| | |
|---|---|
| Cocoa liquor | 1580 parts; |
| Cocoa butter | 800 parts; |
| Maltitol | 960 parts; |
| Xylitol | 241 parts; |
| Lecithin | 16 parts; |
| Stevioside | 3 parts; |
| Traditional Chinese medicine extract | 400 parts. |

In some embodiments, the chocolate 3D printing material comprises the following raw materials in parts by mass:

| | |
|---|---|
| Cocoa liquor | 1495 parts; |
| Cocoa butter | 712 parts; |
| Maltitol | 870 parts; |
| Xylitol | 290 parts; |
| Lecithin | 28 parts; |
| Stevioside | 5 parts; |
| Traditional Chinese medicine extract | 600 parts. |

The chocolate 3D printing material provided by the present disclosure includes traditional Chinese medicine extract. Traditional Chinese medicine extract has some health-care functions, such as regulating blood sugar and blood lipid, enhancing immunity, anti-oxidation, anti-aging, etc. More importantly, traditional Chinese medicine extract is in the forms of particles or powders. The particles contain a crude fiber structure with pores on the surface, and this spatial network structure limits the migration of dispersed phase. The polysaccharides, proteins, and celluloses rich in traditional Chinese medicine extract have a function of emulsification to increase mutual miscibility of the complex multiphase systems of chocolate. Also, the particles of traditional Chinese medicine extract work as a crystal nucleus during chocolate printing, which can induce the formation of a stable crystal form V, improving the taste and appearance of the printed chocolate product. Moreover, the traditional Chinese medicine extract may effectively prevent chocolate from fat blooming and extend the shell life of the chocolate.

In some embodiments, the traditional Chinese medicine in the traditional Chinese medicine extract is at least one selected from POLYGONATI RHIZOMA, LENTINULA EDODES, TREMELLA FUCIFORMIS, LYCII FRUCTUS, PORIA, GANODERMA LUCIDUM, GINKGO FOLIUM, FLAMMULINA VELUTIPES, CUSCUTAE SEMEN, OPHIOPOGONIS RADIX, GLYCYRRHIZAE RADIX ET RHIZOMA, MORI FRUCTUS, CASSIAE SEMEN, CHRYSANTHEMI FLOS, and NOTOGINSENG RADIX ET RHIZOMA.

In some embodiments, the traditional Chinese medicine consists of LENTINULA EDODES, PORIA, FLAMMULINA VELUTIPES, TREMELLA FUCIFORMIS, LYCII FRUCTUS, MORI FRUCTUS, CASSIAE SEMEN, and CHRYSANTHEMI FLOS.

In some embodiments, the traditional Chinese medicine consists of GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA, and GLYCYRRHIZAE RADIX ET RHIZOMA.

In some embodiments, the traditional Chinese medicine extract is composed of an extract A and an extract B; wherein the traditional Chinese medicine in the extract A consists of LENTINULA EDODES, PORIA, FLAMMULINA VELUTIPES, TREMELLA FUCIFORMIS, LYCII FRUCTUS, MORI FRUCTUS, CASSIAE SEMEN, and CHRYSANTHEMI FLOS; wherein the traditional Chinese medicine in the extract B consists of GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA, and GLYCYRRHIZAE RADIX ET RHIZOMA. The mass ratio of extract A and extract B is 2:1. In the chocolate 3D printing material provided by the present disclosure, the traditional chocolate sugar is replaced with low energy maltitol and xylitol, such as sucrose and glucose, which lowers the energy and maintains the original rich flavor, quality and unique taste of chocolate. More importantly, as the ratio of maltitol and xylitol is around 3:1, its effect on the rheological properties of the chocolate is similar to that of sucrose, which may improve the thermal stability of chocolate storage, and may stable the viscosity of the chocolate paste, so that ensuring the smooth discharge during 3D printing.

Lecithin acts as an emulsifier, and stevioside may further adjust the taste and promote the formation of chocolate.

The method for producing the chocolate 3D printing material provided by the present disclosure comprises:

melting the cocoa liquor at 40˜50° C., subjecting the melted cocoa, maltitol, xylitol, and the traditional Chinese medicine extract to rough grinding at 30˜32° C. for 3˜5 h;

adding ⅙˜¾ of the formula amount of the cocoa butter and carrying out fine grinding at 34˜36° C. for 5˜7 h;

adding ⅙˜¾ of the formula amount of the coca butter and carrying out refining at 50˜60° C. for 8˜12 h;

adding lecithin, stevioside, and ⅙˜¾ of the formula amount of the coca butter and carrying out emulsification at 32˜33° C. for about 3˜5 h; and

cooling down the resultant to 25˜35° C., and forming a solid chocolate 3D printing material by marble tempering.

In some embodiments, the method for producing the chocolate 3D printing material comprises:

melting the cocoa liquor at 45° C., subjecting the melted cocoa, maltitol, xylitol, and the traditional Chinese medicine extract to rough grinding at 31° C. for 4 h;

adding ⅙˜¾ of the formula amount of the cocoa butter and carrying out fine grinding at 35° C. for 6 h;

adding ⅙˜¾ of the formula amount of the coca butter and carrying out refining at 55° C. for 10 h;

adding lecithin, stevioside, and ⅙˜¾ of the formula amount of the coca butter and carrying out emulsification at 32˜33° C. for about 4 h; and

cooling down the resultant to 30° C., and forming the solid chocolate 3D printing material by marble tempering.

In the present disclosure, the rough grinding and fine grinding are performed by ball mill, and the movement of the ball can increase the miscibility between the materials and decrease the particle size of the material. On the one hand, it improves the smooth taste of the chocolate, and on the other hand, it can be used for 3D printing without blocking holes to ensure the smooth discharge during the printing. Additionally, the chocolate produced by 3D printing achieves the diversification of the chocolate creative production to satisfy the individualized customization of people, and provides more choices for the chocolate market. The whole production process is carried out in one ball mill, which integrates rough grinding, fine grinding, refining, and emulsification in one machine step by step continuously, which avoids the waste of materials by multi-step and multi-machine, and is suitable for small volume of individualized production.

The present disclosure further provides a method for producing 3D printed chocolate, comprising printing the 3D printed chocolate by a chocolate 3D printer using the chocolate 3D printing material provided by the present disclosure.

In the present disclosure, the temperature of the printing is 28˜33° C., and the printing speed is 5˜24 mm/s.

Preferably, the printing temperature is 30° C.

The 3D printer is a ChocEdge-II chocolate 3D printer.

The present disclosure further provides a 3D printed chocolate, which is produced by the chocolate 3D printing material of the present disclosure.

The present disclosure provides a chocolate 3D printing material and a method for producing the same. In the chocolate 3D printing material provided by the present disclosure, the conventional chocolate sugar is replaced with maltitol and xylitol, which increases the fluidity of the chocolate paste while ensures the taste, and guarantees smooth discharge during 3D printing. The application of the traditional Chinese medicine extract may induce the formation of a stable crystal form V, promoting the mutual miscibility of chocolate multiphase system, preventing chocolate bloom, and extending the shell life of chocolate.

### BRIEF DESCRIPTION OF DRAWINGS

FIG. 1 shows the rheological test results of the chocolate paste, wherein FIG. 1-a shows the full data graph, FIG. 1-b is a partially enlarged schematic view under the shearing rate of 3˜14.

FIG. 2 shows the two-dimensional pattern printed by using the raw materials prepared in Example 2.

FIG. 3 shows the three-dimensional pattern printed by using the raw materials prepared in e Example 3.

### DETAILED DESCRIPTION

The present disclosure provides a chocolate paste material for 3D printing and a method for producing the same. Those skilled in the art may learn from the contents from this document and appropriately improve the processing parameters to produce. Specifically, it should be noted that all such similar alternatives and modifications are obvious to those skilled in the art and are considered to be included in the present disclosure. The method and the application of the present disclosure have been described in the preferred embodiments, and it is obvious to those skilled in the art that the method and application of the present disclosure may be modified or appropriate changed and combined to achieve and apply the present disclosure without departing from the spirit and scope of the present disclosure.

The materials used in the present disclosure are all commercially available products, which can be purchased from the market.

Wherein, the traditional Chinese medicine extract extracted from LENTINULA EDODES, PORTA, FLAMMULINA VELUTIPES, TREMELLA FUCIFORMIS, LYCII FRUCTUS, MORI FRUCTUS, CASSIAE SEMEN, and CHRYSANTHEMI FLOS together is an aqueous extract, and the method for producing the aqueous extract is referred to the Chinese patents CN201410273728.1 and CN105747232A.

The traditional Chinese medicine extract extracted from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA, and GLYCYRRHIZAE RADIX ET RHIZOMA together is an aqueous extract, and the method for producing the aqueous extract is referred to the Chinese patent CN200510014592.3.

The present disclosure is further illustrated below in conjunction with examples:

### Example 1

1) The raw materials were measured according to the following weights: cocoa liquor 1580 g, cocoa butter 800 g, maltitol 1260 g, xylitol 341 g, lecithin 16 g, and stevioside 3 g.

2) The cocoa liquor was melted at 45° C. and added to a ball mill maintained at 30° C.

3) Powder materials such as maltitol and xylitol were added to the ball mill, and the system temperature was controlled at 30˜32° C. by interlayer circulating water, then rough grinding was carried out for 4 h.

4) 260 g of cocoa butter was added to the ball mill, and the system temperature was controlled at 34˜36° C. by interlayer circulating water, then fine grinding was carried out for 6 h.

5) 240 g of cocoa butter was added to the ball mill, and the system temperature was controlled at 54° C., then refining was carried out for about 10 h.

6) 300 g of cocoa butter, 16 g of lecithin and 3 g of stevioside were added to the ball mill, and the system temperature was controlled at 32˜33° C., then emulsification was carried out for about 4 h to obtain a chocolate paste.

7) The temperature of the chocolate paste in step 6) was cooled down to 30° C., and a solid printing material was formed by marble tempering.

### Example 2

1) The raw materials were measured according to the following weights: cocoa liquor 1580 g, cocoa butter 800 g, 400 g dry powder of the compound aqueous extract from LENTINULA EDODES, PORIA, FLAMMULINA VELUTIPES, TREMELLA FUCIFORMIS, LYCII FRUCTUS, MORI FRUCTUS, CASSIAE SEMEN, and CHRYSANTHEMI FLOS, maltitol 960 g, xylitol 241 g, lecithin 16 g, and stevioside 3 g.

2) The cocoa liquor was melted at 45° C. and added to a ball mill maintained at 30° C.

3) Powder materials such as the extract from LENTINULA EDODES, PORIA, FLAMMULINA VELUTIPES, TREMELLA FUCIFORMIS, LYCII FRUCTUS, MORI FRUCTUS, CASSIAE SEMEN and CHRYSANTHEMI FLOS, maltitol, and xylitol were added to the ball mill, and the system temperature was controlled at 30˜32° C. by interlayer circulating water, then rough grinding was carried out for 4 h.

4) 260 g of cocoa butter was added to the ball mill, and the system temperature was controlled at 34˜36° C. by interlayer circulating water, then fine grinding was carried out for 6 h.

5) 240 g of cocoa butter was added to the ball mill, and the system temperature was controlled at 54° C., then refining was carried out for about 10 h.

6) 300 g of cocoa butter, 16 g of lecithin and 3 g of stevioside were added to the ball mill, and the system temperature was controlled at 32˜33° C., then emulsification was carried out for about 4 h to obtain a chocolate paste.

7) The temperature of the chocolate paste in step 6) was cooled down to 30° C., and a solid printing material was formed by marble tempering.

### Example 3

1) The raw materials were measured according to the following weights: cocoa liquor 1495 g, cocoa butter 712 g, 400 g dry powder of the compound aqueous extract from LENTINULA EDODES, PORIA, FLAMMULINA VELUTIPES, TREMELLA FUCIFORMIS, LYCII FRUCTUS, MORI FRUCTUS, CASSIAE SEMEN and CHRYSANTHEMI FLOS, 200 g dry powder of the compound aqueous extract from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA and GLYCYRRHIZAE RADIX ET RHIZOMA, maltitol 870 g, xylitol 290 g, lecithin 28 g, and stevioside 5 g.

2) The cocoa liquor was melted at 45° C. and added to a ball mill maintained at 30° C.

3) Powder materials such as the extract from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA and GLYCYRRHIZAE, maltitol, and xylitol were added to the ball mill, and the system temperature was controlled at 30˜32° C. by interlayer circulating water, then rough grinding was carried out for 4 h.

4) 220 g of cocoa butter was added to the ball mill, and the system temperature was controlled at 34˜36° C. by interlayer circulating water, then fine grinding was carried out for 6 h.

5) 240 g of cocoa butter was added to the ball mill, and the system temperature was controlled at 54° C., then refining was carried out for about 10 h.

6) 252 g of cocoa butter, 28 g of lecithin and 5 g of stevioside were added to the ball mill, and the system temperature was controlled at 32˜33° C., then emulsification was carried out for about 4 h to obtain a chocolate paste.

7) The temperature of the chocolate paste in step 6) was cooled down to 30° C., and a solid printing material was formed by marble tempering.

### Example 4

1) The raw materials were measured according to the following weights: cocoa liquor 1200 g, cocoa butter 1200 g, 400 g dry powder of the compound aqueous extract from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA and GLYCYRRHIZAE RADIX ET RHIZOMA, maltitol 902 g, xylitol 277 g, lecithin 20 g, and stevioside 4 g.

2) The cocoa liquor was melted at 45° C. and added to a ball mill maintained at 30° C.

3) Powder materials such as the extract from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA and GLYCYRRHIZAE, maltitol, and xylitol were added to the ball mill, and the system temperature was controlled at 30˜32° C. by interlayer circulating water, then rough grinding was carried out for 4 h.

4) 200 g of cocoa butter was added to the ball mill, and the system temperature was controlled at 34˜36° C. by interlayer circulating water, then fine grinding was carried out for 6 h.

5) 200 g of cocoa butter was added to the ball mill, and the system temperature was controlled at 54° C., then refining was carried out for about 10 h.

6) 800 g of cocoa butter, 20 g of lecithin and 4 g of stevioside were added to the ball mill, and the system temperature was controlled at 32˜33° C., then emulsification was carried out for about 4 h to obtain a chocolate paste.

7) The temperature of the chocolate paste in step 6) was cooled down to 30° C., and a solid printing material was formed by marble tempering.

### Example 5

1) The raw materials were measured according to the following weights: cocoa liquor 1120 g, cocoa butter 600 g, 400 g dry powder of the compound aqueous extract from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA and GLYCYRRHIZAE RADIX ET RHIZOMA, maltitol 1335 g, xylitol 520 g, lecithin 24 g, and stevioside 1 g.

2) The cocoa liquor was melted at 45° C. and added to a ball mill maintained at 30° C.

3) Powder materials such as the extract from GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA and GLYCYRRHIZAE, maltitol, and xylitol were added to the ball mill, and the system temperature was controlled at 31˜33° C. by interlayer circulating water, then rough grinding was carried out for 4 h.

4) 220 g of cocoa butter was added to the ball mill, and the system temperature was controlled at 33˜35° C. by interlayer circulating water, then fine grinding was carried out for 6 h.

5) 180 g of cocoa butter was added to the ball mill, and the system temperature was controlled at 58° C., then refining was carried out for about 10 h.

6) 200 g of cocoa butter, 24 g of lecithin and 1 g of stevioside were added to the ball mill, and the system temperature was controlled at 34° C., then emulsification was carried out for about 4 h to obtain a chocolate paste.

7) The temperature of the chocolate paste in step 6) was cooled down to 30° C., and a solid printing material was formed by marble tempering.

### Example 6

1) The raw materials were measured according to the following weights: cocoa liquor 1580 g, cocoa butter 800 g, 200 g ultrafine microcrystalline cellulose with a diameter of 0.05˜0.1 μm and a length of 0.3˜0.8 μm, maltitol 1110 g, xylitol 291 g, lecithin 16 g, and stevioside 3 g.

2) The cocoa liquor was melted at 45° C. and added to a ball mill maintained at 30° C.

3) Powder materials such as cellulose, maltitol and xylitol were added to the ball mill, and the system temperature was controlled at 30˜32° C. by interlayer circulating water, then rough grinding was carried out for 4 h.

4) 240 g of cocoa butter was added to the ball mill, and the system temperature was controlled at 34˜36° C. by interlayer circulating water, then fine grinding was carried out for 6 h.

5) 260 g of cocoa butter was added to the ball mill, and the system temperature was controlled at 54° C., then refining was carried out for about 10 h.

6) 300 g of cocoa butter, 16 g of lecithin and 3 g of stevioside were added to the ball mill, and the system temperature was controlled at 32˜33° C., then emulsification was carried out for about 4 h to obtain a chocolate paste.

7) The temperature of the chocolate paste in step 6) was cooled down to 30° C., and a solid printing material was formed by marble tempering.

### Example 7

ChocEdge-II chocolate 3D printer was preferred in the present disclosure. The chocolate raw materials prepared in examples 1˜6 were melted at 45° C., and then unmelted material was added to printer at ⅓ of the melted amount for use as a seed crystal. The forming is evaluated by recording the complete solidification time of printing a layer of 5 cm×5 cm square frame and the number of the stackable layers of 5 cm×5 cm square frame.

TABLE 1
Printing formability of the chocolate materials prepared in the examples

| | Complete solidification time of | Continuously printing |
|---|---|---|
| Example | the first layer/min | stackable layers |
| 1 | 3.5 | 20.6 ± 4.3 |
| 2 | 3.1 | ≥50* |
| 3 | 3.3 | ≥50* |
| 4 | — | — |
| 5 | — | — |
| 6 | 3.4 | 36.8 ± 5.1 |

*Note:
The maximum printing height of ChocEdge-II chocolate 3D printer is 5 cm, and the single layer height is 1 mm during the printing and the limit is 50 layers.

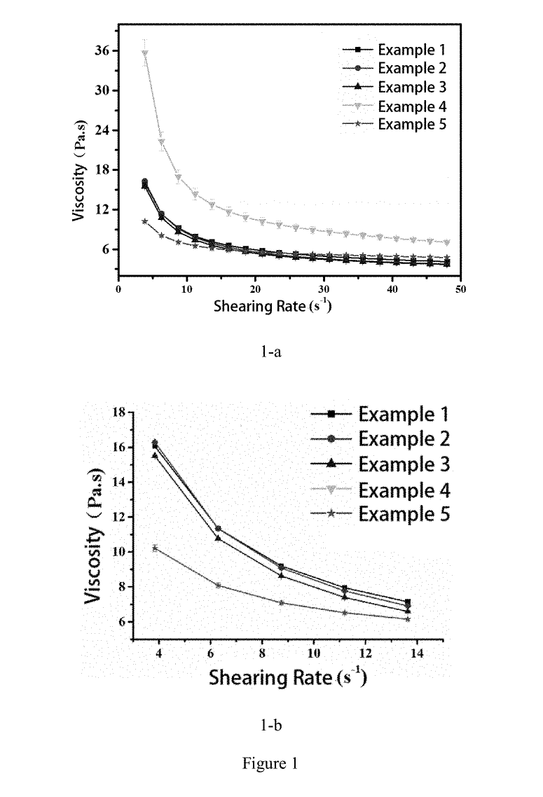

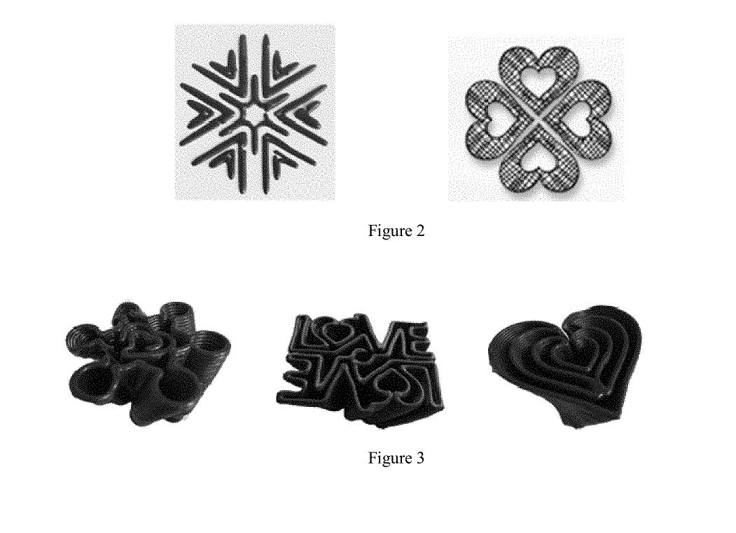

The results showed that the chocolate raw materials of examples 1˜3 have a good formability, and the solidification time of the single layer was shortened, and the number of layers that can be effectively deposited was high. However, examples 4˜5 could not form chocolate layer. From the rheological data analysis in FIG. 1, it can be seen that the melt viscosity of examples 1˜3 fell within a relatively proper range, which is applicable to 3D printer processing. The ratio of the cocoa butter and cocoa liquor was too high in the formula of Example 4, and the viscosity of the paste was too low, thus the paste is turbulent when printing, and the target shape might not be printed. The ratio of the cocoa butter and cocoa liquor was too low in the formula of Example 5, and the viscosity of the paste was too high, so the extruded line was deformed, and the target shape might not be printed. Examples 2˜3 were significantly higher than Example 1 and Example 6 in terms of stackable layers, p<0.05.

### Example 8

ChocEdge-II chocolate 3D printer was preferably used to print chocolate and the printed samples were stored in a PET box at 25° C., and the appearance and taste of printed chocolate placed for different time periods were evaluated.

Crowd scoring experiment was used to evaluate the appearance and taste. The chocolate samples were printed at a size of 20×20×6 mm and the test environment conditions were kept constant at 22˜24° C. In order to reduce the influence of many factors, such as hobbies and preferences, from the measurement to the formation of the concept, on the test results, the test was performed by double-blind method. That is, the samples were coded with three random numbers in this study, and the test samples were also randomized. The rating score was 100 points, and the basic rating criteria were shown in the table below. 10 college students were invited to form an assessment team, and the purpose of the test and the indicators and precautions for the evaluation were first clarified. Each evaluation was carried out independently by each of the assessed members, and they were not in contact with each other. The mouth was rinsed with water after finishing each evaluation. The rating criteria are as shown in Table 2:

TABLE 2
Taste and appearance rating criteria

| | Evaluation | | Rating criteria | | |
|---|---|---|---|---|---|
| Type | project | Points | 7~10 | 4~6 | 0~3 |
| Appearance | Glossiness | 10 | Uniform, glossy | Dim gloss | Matte |
| | Color degree | 10 | Uniform color, | Partial chromatic | Speckled |
| | | | no floating | aberration | |
| | Fracture | 10 | Compact structure, | Tiny pores | Obvious pores |
| | surface | | no visible pores | | and particles |
| | compactness | | | | |
| Odor | Chocolate | 10 | Moderate aroma | General | Strong aroma |
| | aroma | | | | or no aroma |
| | Extract odor | 10 | No obvious smell | Acceptable | Unpleasant smell |
| Taste | Solubility | 10 | Just melted | Melted within | Unmelted within |
| | in mouth | | in the mouth | 5 s | 5 s |
| | Delicateness | 10 | Non sticky tongue | Sticky tongue or | Rough tates, felt |
| | | | | slightly felt grainy | grainy |
| | Greasiness | 10 | Not greasy | General | Greasy |
| | Hardness | 10 | Moderate | General | Too soft or |
| | | | | | too hard |
| | Sweetness | 10 | Moderate | General | Too sweet or |
| | | | | | too bitter |

The evaluation results are shown in Table 3:

TABLE 3
Appearance and taste rating criteria of chocolate printing materials

| | 24 h | | | 72 h | | | 7 days | | |
|---|---|---|---|---|---|---|---|---|---|
| | Ap- | | | Ap- | | | Ap- | | |
| Exam- | pear- | | | pear- | | | pear- | | |
| ples | ance | Odor | Taste | ance | Odor | Taste | ance | Odor | Taste |
| 1 | 26.1 | 17.4 | 43.5 | 22.8 | 15.2 | 38.0 | 18.9 | 12.6 | 31.5 |
| 2 | 27.6 | 18.4 | 46.0 | 25.8 | 17.2 | 43.0 | 22.2 | 14.8 | 37.0 |
| 3 | 27.0 | 18.2 | 45.3 | 25.2 | 16.8 | 42.0 | 21.9 | 14.6 | 36.5 |

Chocolate is often stored at 18˜20° C., but considering that the crystal form V of chocolate is more likely to change to the crystal form VI at 25° C., a short-term storage experiment was performed at 25° C. Appearance and taste evaluation experiments showed that the chocolate has a longer lasting scent, a better gloss and an improved taste after adding the extract. The scores of examples 2˜3 were significantly higher than those of Example 1, p<0.05.

### Example 9

The prepared chocolate raw material was printed into a size of 20×20×6 mm and packaged in an aluminum foil bag, was and then stored in an incubator at 25° C. with a humidity≈50%. The appearance and taste evaluation were carried out after different time periods, and the appearance and taste evaluation methods were the same as in Example 8. The evaluation results are shown in Table 4.

TABLE 4
Appearance and taste evaluation of chocolate printing
materials after different time periods

| | 1 month | | | 3 months | | | 12 months | | |
|---|---|---|---|---|---|---|---|---|---|
| | Ap- | | | Ap- | | | Ap- | | |
| Exam- | pear- | | | pear- | | | pear- | | |
| ples | ance | Odor | Taste | ance | Odor | Taste | ance | Odor | Taste |
| 1 | 26.7 | 17.8 | 44.5 | 19.8 | 13.2 | 33.2 | 17.4 | 11.6 | 29.2 |
| 2 | 27.5 | 18.3 | 46.1 | 22.2 | 14.8 | 37.1 | 19.2 | 12.8 | 32.3 |
| 3 | 27.3 | 18.2 | 45.5 | 21.6 | 14.4 | 36.3 | 18.6 | 12.4 | 31.2 |
| 6 | 26.9 | 18.0 | 45.4 | 20.4 | 13.6 | 34.4 | 17.7 | 11.8 | 29.5 |

After storage, the crystal form of the chocolate often changes and the excessive crystal form VI makes the chocolate grainy. Crystal form VI has a higher melting point, so the solubility in mouth is worse; and a various of crystal forms in the chocolate reduce its silky feeling, give a dim surface, and even the fat is bloomed with white spots. The results showed that the appearance and taste of the chocolates made from each printing materials decreased after the storage. However, after 12 months of storage, the properties of examples 2˜3 were still significantly better than that of examples 1 and 6, p<0.05. It indicates that the addition of traditional Chinese medicine extract has a positive impact on the chocolate properties.

### Example 10

The prepared chocolate raw materials were packaged in an aluminum foil bag and stored in an incubator at 18° C. with a humidity≈50%. After different time periods, part of the raw materials was taken out for printing, and the printability after storage was evaluated.

TABLE 5
Printability evaluation of chocolate printing
materials after different time periods

| | The number of stackable layers | | |
|---|---|---|---|
| Examples | 1 month | 3 months | 12 months |
| 1 | 19.8 ± 4.2 | 14.4 ± 4.7 | 7 ± 5.8 |
| | | | (blocking hole and stopping) |
| 2 | ≥50* | ≥50* | ≥50* |
| 3 | ≥50* | ≥50* | ≥50* |
| 6 | 36.3 ± 4.7 | 32.5 ± 4.6 | 22 ± 6.7 |
| | | | (blocking hole and stopping) |

*Note:
The maximum printing height of ChocEdge-II chocolate 3D printer is 5 cm, and the single layer height is 1 mm during the printing and the limit is 50 layers.

The results showed that the printing materials of examples 2˜3 still can be printed smoothly after storage, and the number of stackable layers did not change significantly, which might meet the printing requirements. Due to the migration of grease and the aggregation of sugar in Example 1 and Example 6 during the storage, partial material became uneven, resulting in a significant decrease in the number of stackable layers, demonstrating that the formulas of examples 2 and 3 are more reasonable.

### Example 11

The prepared chocolate raw materials were put into a corresponding cartridge of ChocEdge-II chocolate 3D printer and packed in an aluminum foil bag, and then stored in an incubator at 18° C. with a humidity≈50%. The cartridge was taken out after different time periods, melted at 37° C., and then continuously extruded in the printer. The printability after storage was evaluated by weighing the continuous extrusion output.

TABLE 5
Printability evaluation of chocolate printing
materials after different time periods

| | Continuous extrusion mass during printing/g | | |
|---|---|---|---|
| Examples | 1 month | 3 months | 12 months |
| 1 | 32.6 ± 0.7 | 29.3 ± 1.6 | 17.2 ± 5.2 |
| 2 | 34.1 ± 0.6* | 33.4 ± 0.6* | 33.8 ± 0.7* |
| 3 | 33.7 ± 0.5* | 33.6 ± 0.4* | 33.2 ± 1.2* |
| 6 | 33.6 ± 0.7 | 28.3 ± 1.7 | 16.1 ± 4.6 |

*Note:
ChocEdge-II chocolate 3D printer cartridge can hold up to 35 g of material at a time, and usually about 34 g can be smoothly extruded.

The results showed that the printing materials of examples 2˜3 still were extruded smoothly after storage without blocking the hole. However, due to the migration of grease and the aggregation of dispersed sugar particles in Example 1 and Example 6 during storage, partial material became uneven, resulting in hole blocking and a significant reduction of the continuous extrusion mass, demonstrating that the formulas of examples 2 and 3 are more reasonable.

The above are only preferred examples of the present disclosure, and it should be noted that those skilled in the art may also make several improvements and modifications without departing from the principles of the present disclosure. These improvements and modifications should also be considered as protection scope of the present disclosure.

What is claimed is:

1. A chocolate 3D printing material, which is made from the following raw materials in parts by mass:

| | |
|---|---|
| Cocoa liquor | 1495-1580 parts; |
| Cocoa butter | 712-800 parts; |
| Maltitol | 870-1260 parts; |
| Xylitol | 241-341 parts; |
| Lecithin | 16-28 parts; |
| Stevioside | 1-5 parts; |
| Traditional Chinese medicine extract | 0-600 parts. |

2. The chocolate 3D printing material according to claim 1, which is made from the following raw materials in parts by mass:

| | |
|---|---|
| Cocoa liquor | 1495-1580 parts; |
| Cocoa butter | 712-800 parts; |
| Maltitol | 870-960 parts; |
| Xylitol | 241-290 parts; |
| Lecithin | 16-28 parts; |
| Stevioside | 3-5 parts; |
| Traditional Chinese medicine extract | 400-600 parts. |

3. The chocolate 3D printing material according to claim 1, wherein the traditional Chinese medicine in the traditional Chinese medicine extract is at least one selected from POLYGONATI RHIZOMA, LENTINULA EDODES, TREMELLA FUCIFORMIS, LYCII FRUCTUS, PORTA, GANODERMA LUCIDUM, GINKGO FOLIUM, FLAMMULINA VELUTIPES, CUSCUTAE SEMEN, OPHIOPOGONIS RADIX, GLYCYRRHIZAE RADIX ET RHIZOMA, MORI FRUCTUS, CASSIAE SEMEN, CHRYSANTHEMI FLOS, and NOTOGINSENG RADIX ET RHIZOMA.

4. The chocolate 3D printing material according to claim 3, wherein the traditional Chinese medicine consists of LENTINULA EDODES, PORIA, FLAMMULINA VELUTIPES, TREMELLA FUCIFORMIS, LYCII FRUCTUS, MORI FRUCTUS, CASSIAE SEMEN, and CHRYSANTHEMI FLOS.

5. The chocolate 3D printing material according to claim 3, wherein the traditional Chinese medicine consists of GANODERMA LUCIDUM, ATRACTYLODIS MACROCEPHALAE RHIZOMA, POLYGONATI RHIZOMA, LYCII FRUCTUS, CUSCUTAE SEMEN, OPHIOPOGONIS RADIX, PORIA, and GLYCYRRHIZAE RADIX ET RHIZOMA.

6. A method for producing the chocolate 3D printing material according to claim 1, comprising:
melting the cocoa liquor at 40˜50° C., subjecting the melted cocoa, maltitol, xylitol, and the traditional Chinese medicine extract to rough grinding at 30˜32° C. for 3˜5 h;
adding ⅙˜¾ of the formula amount of the cocoa butter and carrying out fine grinding at 34˜36° C. for 5˜7 h;
adding ⅙˜¾ of the formula amount of the coca butter and carrying out refining at 50˜60° C. for 8˜12 h;
adding lecithin, stevioside, and ⅙˜¾ of the formula amount of the coca butter and carrying out emulsification at 32˜33° C. for about 3˜5 h; and
cooling down the resultant to 25˜35° C., and forming a solid chocolate 3D printing material by marble tempering.

7. The method according to claim 6, comprising:
melting the cocoa liquor at 45° C., subjecting the melted cocoa, maltitol, xylitol, and the traditional Chinese medicine extract to rough grinding at 31° C. for 4 h;
adding ⅙˜¾ of the formula amount of the cocoa butter and carrying out fine grinding at 35° C. for 6 h;
adding ⅙˜¾ of the formula amount of the coca butter and carrying out refining at 55° C. for 10 h;
adding lecithin, stevioside, and ⅙˜¾ of the formula amount of the coca butter and carrying out emulsification at 32˜33° C. for about 4 h; and
cooling down the resultant to 30° C., and forming the solid chocolate 3D printing material by marble tempering.

8. A method for producing a 3D printed chocolate, comprising using the chocolate 3D printing material according to claim 1 to print the 3D printed chocolate by a chocolate 3D printer.

9. The method according to claim 8, wherein the temperature of the printing is 28˜33° C., and the printing speed is 5˜24 mm/s.

10. A 3D printed chocolate, which is produced by using the chocolate 3D printing material according to claim 1.
